(19) United States
(12) Patent Application Publication
(10) Pub. No.: US 2013/0079682 A1
(43) Pub. Date: Mar. 28, 2013

(54) ULTRASOUND-NEUROMODULATION TECHNIQUES FOR CONTROL OF PERMEABILITY OF THE BLOOD-BRAIN BARRIER

(71) Applicant: David J. Mischelevich, Playa del Rey, CA (US)

(72) Inventor: David J. Mischelevich, Playa del Rey, CA (US)

(21) Appl. No.: 13/625,677

(22) Filed: Sep. 24, 2012

Related U.S. Application Data
(60) Provisional application No. 61/538,934, filed on Sep. 25, 2011.

(51) Int. Cl. A61N 7/00; A61M 37/00

(52) U.S. Cl. CPC A61N 7/00; A61M 37/0092
USPC 601/2; 604/22

(57) ABSTRACT

Disclosed are methods and systems and methods employing non-invasive ultrasound-neuromodulation techniques to control the permeability of the blood-brain barrier. For example, such an alteration can permit increased penetration of a medication to increase its therapeutic effect. The neuromodulation can produce acute or long-term effects. The latter occur through Long-Term Depression (LTD) and Long-Term Potentiation (LTP) via training. Included is control of direction of the energy emission, intensity, frequency (carrier and/or neuromodulation frequency), pulse duration, firing pattern, and phase/intensity relationships for beam steering and focusing on targets and accomplishing up-regulation and/or down-regulation.

### CROSS REFERENCE TO RELATED APPLICATIONS

This application claims priority to Provisional Patent Application No. 61/538,934 (Attorney Docket No. 42927-715.101) filed Sep. 25, 2011, the entire contents of which is incorporated herein in its entirety.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for using ultrasound-neuromodulation techniques for the treatment of medical conditions.

### BACKGROUND OF THE INVENTION

It has been demonstrated that focused ultrasound directed at neural structures can stimulate those structures. If neural activity is increased or excited, the neural structure is up regulated; if neural activated is decreased or inhibited, the neural structure is down regulated. Preliminary clinical work by universities (Ben-Gurion University and the University of Rome) using Brainsway Transcranial Magnetic Stimulation (TMS) systems has shown that deep-brain neuromodulation can open up the blood-brain barrier to allow more effective penetration of drugs (e.g., for the treatment of malignant tumors). Ultrasound would be more effective for this purpose because of its higher resolution and thus more specificity. The equipment also costs less and can be portable for use in a variety of settings, including within the home of the patient.

Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well. He describes a mechanism for neuromodulation caused by an ultrasonic wave that induces particle motion and thus an electric current density via Lorentz forces.

The effect of ultrasound on neural activity is at least two fold according to the present invention. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound mediated opening of calcium channels was also observed by Tyler and colleagues. The above approach is incorporated in a patent application submitted by Tyler (Tyler, William, James P., PCT/US2009/050560, WO 2010/009141, published Jan. 21, 2011).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap placed over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med, Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as typically below 500 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. patent application Ser. No. 12/263,026 published as US 2009/0112133 A1 Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as compared to TMS that can be focused to 1 cm at best.

Because of the utility of ultrasound in the neuromodulation of deep-brain structures, application of those techniques to alteration of the permeability of the blood-brain barrier is both logical and desirable even though the target is the blood-brain barrier and not necessarily involving the neuromodulation of the neural target itself.

### SUMMARY OF THE INVENTION

It is the purpose of this invention to provide methods and systems using non-invasive ultrasound-neuromodulation techniques to selectively alter the permeability of the blood-brain barrier (either brain or spinal cord). Early work at Ben-Gurion University and the University of Rome using Brainsway in Transcranial Magnetic Stimulation (TMS) systems has shown that deep-brain neuromodulation techniques can alter the permeability of the blood-brain barrier to allow more effective penetration of drugs (e.g., for the treatment of malignant tumors). Tumors to which opening of the blood-brain barrier using other techniques has been applied are gliomas, CNS lymphoma and metastatic cancer to the brain. The equipment employed in the current invention also costs less and can be portable for use in a variety of settings, including within the home of the patient.

Such neuromodulation can produce acute effects or Long-Term Potentiation (LTP) or Long-Term Depression (LTD). Included is control of direction of the energy emission, intensity, frequency (carrier and/or neuromodulation frequency), pulse duration, firing pattern, and phase/intensity relationships for beam steering and focusing on targets and accomplishing up-regulation and/or down-regulation. Use of ancillary monitoring or imaging to provide feedback is optional. In embodiments where concurrent imaging is performed, the device of the invention is constructed of non-ferrous material.

Multiple targets can be neuromodulated singly or in groups to control the permeability of the blood-brain barrier. To accomplish the treatment, in some cases the neural targets will be up regulated and in some cases down regulated, depending on the given target. The targeting can be done with one or more of known external landmarks, an atlas-based approach or imaging (e.g., fMRI or Positron Emission Tomography).

While ultrasound can be focused down to a diameter on the order of one to a few millimeters (depending on the frequency), whether such a tight focus is required depends on the conformation of the target.

### BRIEF DESCRIPTION OF THE DRAWINGS

FIG. 1 shows exemplar blood-brain barrier targets on which ultrasound is focused.

FIG. 2 shows a block diagram of the control circuit.

### DETAILED DESCRIPTION OF THE INVENTION

It is the purpose of this invention to provide methods and systems using non-invasive ultrasound-neuromodulation techniques to selectively alter the permeability of the blood-brain barrier (either brain or spinal cord). If the target is a neural target as opposed to a tumor, the application of the invention may result in effective neuromodulation of that target in addition to altering the permeability of the blood-brain barrier in that region allowing more effective penetration of a drug to impact that neural target. This applies to humans or animals and in brain or spinal cord. The change can control blood-brain permeability by increasing permeability to increase the access of drugs to, for example, neurological targets or tumors or decreasing permeability to protect targets from drugs that could cause side effects. If the application of the techniques results in decreasing the permeability of the blood-brain barrier (in cases where the permeability has been increased through another mechanism), in some cases coincident neuromodulation of a target in the region will have a therapeutic benefit. Multiple conditions are aggravated by breaching of the blood-brain barrier, among which are Alzheimer's Disease, HIV Encephalitis, Multiple Sclerosis, Meningitis, and Epilepsy. Such neuromodulation systems can produce applicable acute or long-term effects. The latter occur through Long-Term Depression (LTD) or Long-Term Potentiation (LTP) via training. Included is control of direction of the energy emission, intensity, frequency (carrier and/or neuromodulation frequency), pulse duration, firing pattern, and phase/intensity relationships for beam steering and focusing on targets and accomplishing up-regulation and/or down-regulation.

What will work for altering the permeability of the blood brain barrier in a given situation depends on a given patient and associated condition. In some situations, excitation will result in increasing the permeability of the blood-brain barrier and inhibition will result in decreasing it. In other situations, the reverse will be true.

Ultrasound is acoustic energy with a frequency above the normal range of human hearing (typically greater than 20 kHz). In this invention, ultrasound-neuromodulation techniques refers to the delivery of ultrasound energy to tissue in the brain or spinal cord having an acoustic frequency in a range of 0.3 MHz to 0.8 MHz with acoustic intensity greater than 20 mW/cm² at the target tissue. The frequency in the range of 0.3 MHz to 0.8 MHz represents the carrier frequency on which amplitude modulation is applied. The amplitude modulation frequency for inhibition or down regulation is typically lower than 500 Hz (depending on condition and patient). The amplitude modulation frequency for excitation is typically in the range of 500 Hz to 5 MHz again depending on condition and patient. In one embodiment, the modulation frequency of lower than approximately 500 Hz is divided into pulses 0.1 to 20 msec. repeated at frequencies of 2 Hz or lower for inhibition or down regulation. In one embodiment, the amplitude modulation frequency of higher than approximately 500 Hz is divided into pulses 0.1 to 20 msec. repeated at frequencies higher than 2 Hz for up regulation. In some embodiments the acoustic intensity is greater than about 30 mW/cm² at the target tissue. The acoustic intensity is less than the appropriate target- or patient-specific levels at which no tissue damage is caused. Ultrasound therapy can be combined with therapy using other devices Transcranial Magnetic Stimulation (TMS)).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space between the ultrasound transducer and the head of a subject.

Altering the permeability of the blood-brain barrier using ultrasound-neuromodulation techniques has significant benefits over other techniques such as Transcranial Magnetic Stimulation neuromodulation (e.g., using the Brainsway system) because ultrasound neuromodulation provides greater resolution and uses hardware that is both less expensive and portable so it can be used at home or other non-clinical-office locations.

A notable benefit is the ability to reduce side effects by having increased permeability in applicable regions where a drug needs to be active and leave at its normal level or decrease permeability in other regions where that drug could cause side effects. This spatial selectivity depends on the ability of the neuromodulation to be selective which is true for ultrasound neuromodulation, but not true for an essentially whole-brain neuromodulation approach such as that of Brainsway or any approach using Transcranial Magnetic Stimulation. Another facet of side effects is the significant opportunity to protect structures by selectively decreasing the permeability in certain regions.

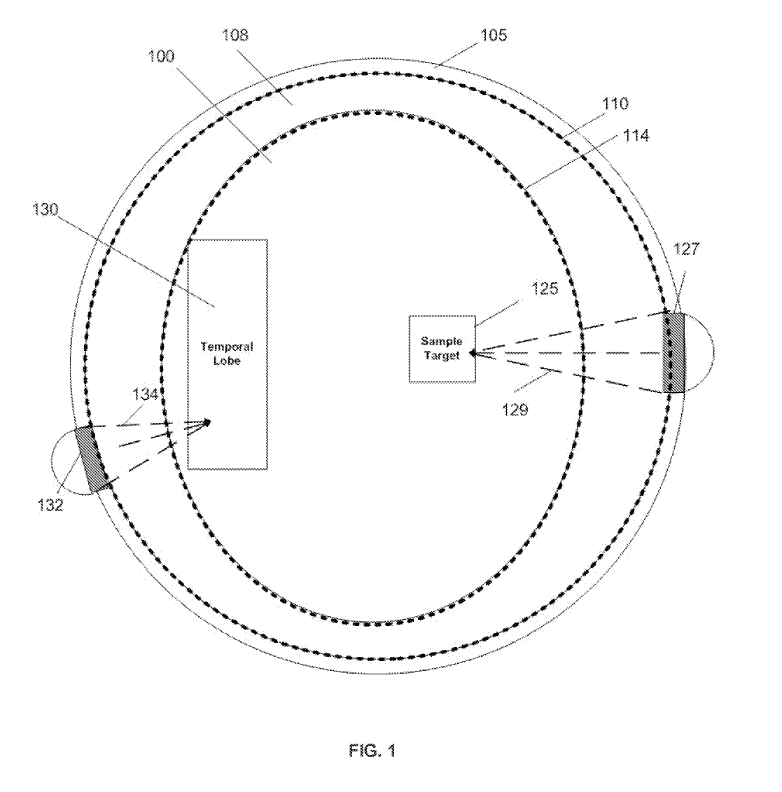

FIG. 1 shows exemplar targets for control of permeability of the blood-brain barrier for the selective penetration of drugs or other substances into the target. Head 100 contains two targets, one a generic Sample Target 125 and the other the Temporal Lobe 130 as an example of a neural target for the treatment of epilepsy. For example, Sample Target 125 may represent a malignant tumor such as glioblastoma multiforme (the subject of the work by Brainsway) to open up the path for anti-tumor drugs and Temporal Lobe 130 would be a target for permeability change to open up the path for anti-epilepsy drugs. There can be different numbers of targets for a given condition and the appropriate targets will change as research evolves. Targets 125 and 130 are targeted by ultrasound from transducers 127 and 132 respectively, fixed to track 105. In other embodiments the ultrasound transducer or transducers can be affixed to the patient's head using other means such as strapping to the head or holding within the framework of a swimming-cap-style structure. Ultrasound transducer 127 with its beam 129 is shown targeting Sample Target 120 and transducer 132 with its beam 134 is shown targeting Temporal Lobe 130. Bilateral stimulation of one of a plurality of these targets is another embodiment. For ultrasound to be effectively transmitted to and through the skull and to brain targets, coupling must be put into place. Ultrasound transmission (for example Dermasol from California Medical Innovations) medium 108 is interposed with one mechanical interface to the frame 105 and ultrasound transducers 127 and 132 (completed by a layer of ultrasound transmission gel layer 110) and the other mechanical interface to the head 100 (completed by a layer of ultrasound transmission gel 114). In another embodiment, the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target. If a large volume of the brain is to have its permeability altered then multiple ultrasound transducers with defocused beams can be employed.

Transducer array assemblies of this type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon and Blatek in the U.S. are other custom-transducer suppliers. The power applied will determine whether the ultrasound is high intensity or low intensity (or medium intensity) and because the ultrasound transducers are custom, any mechanical or electrical. changes can be made, if and as required. At least one configuration available from Imasonic (the HIFU linear phased array transducer) has a center hole for the positioning of an imaging probe. Keramos-Etalon also supplies such configurations.

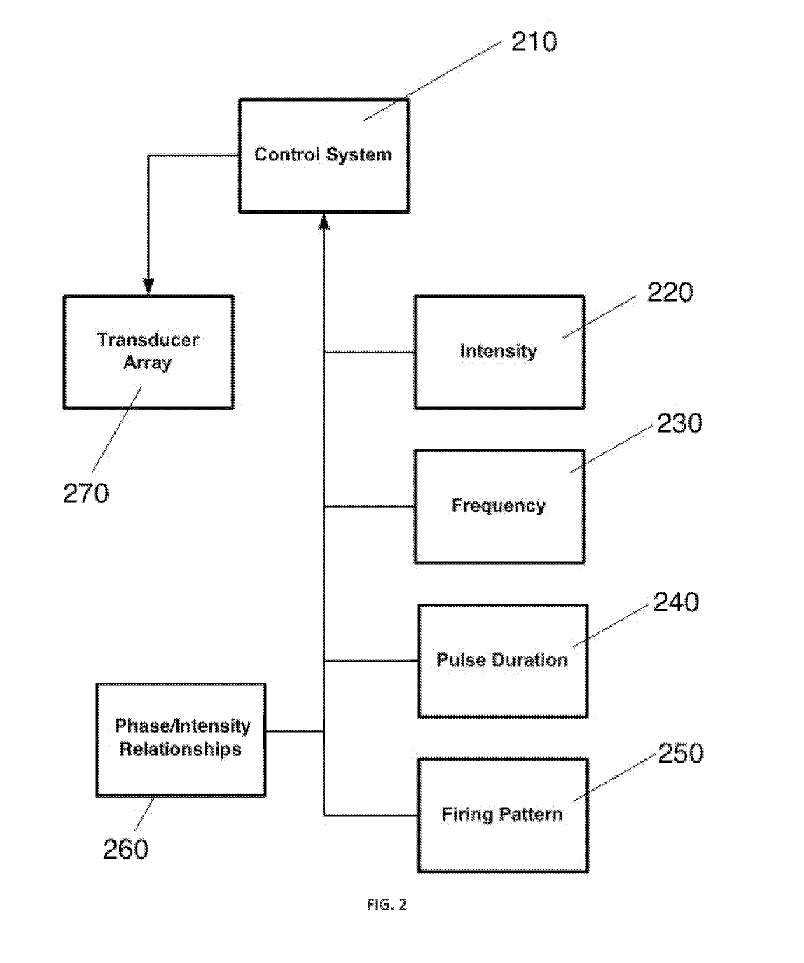

FIG. 2 shows an embodiment of a control circuit. The positioning and emission characteristics of transducer array 270 are controlled by control system 210 with control input with neuromodulation characteristics determined by settings of intensity 220, frequency 230 (can be carrier and/or neuromodulation frequency), pulse duration 240, firing pattern 250, and phase/intensity relationships 460 for beam steering and focusing on neural targets. Instead of phase/frequency relationships that can steer the ultrasound beam, 260 can represent mechanically altering the direction of the ultrasound beam, including axial or radial mechanical perturbations of the ultrasound transducers.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

The invention allows stimulation adjustments in variables such as, but not limited to, intensity, firing pattern, frequency (carrier and/or neuromodulation; frequency), pulse duration, firing pattern, phase/intensity relationships for beam steering, dynamic sweeps, position, and direction, including axial or radial perturbations of the ultrasound transducers.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method for altering a permeability of a blood-brain barrier in a patient, the method comprising:
aiming at least one ultrasound transducer at least one target in a brain or a spinal cord of a human or animal, and
energizing at least one transducer to deliver pulsed ultrasound energy to the at least one target,
wherein permeability of the blood-brain barrier in the vicinity of the target is altered.

2. The method of claim 1 wherein the transducer is controlled to deliver ultrasound pulsed power that increases the permeability of the blood-brain barrier.

3. The method of claim 2 further comprising administering a drug to the patient wherein the effectiveness of the drug is enhanced by increased penetration of that drug into the target because of the increase in permeability of the blood-brain barrier.

4. The method of claim 1 wherein the transducer is controlled to deliver ultrasound pulsed power which decreases the permeability of the blood-brain barrier.

5. The method of claim 4 further comprising administering a drug to the patient wherein the side effects of the drug are reduced due to decreased penetration of the drug into the target because of the decrease in permeability of the blood-brain barrier.

6. The method of claim 1 wherein a target is selected to have permeability to a drug increased to improve the effectiveness of the drug.

7. The method of claim 1 wherein a target is selected to have permeability to a drug decreased to protect the target and decrease the side effects of the drug.

8. The method of claim 1 wherein the ultrasound further provides coincident neuromodulation of a neural target.

9. The method of claim 8 wherein the neuromodulation comprises up-regulation.

10. The method of claim 8 wherein the neuromodulation comprises down-regulation.

11. The method of claim 8 wherein the neuromodulation induces Long-Term Depression.

12. The method of claim 8 wherein the neuromodulation induces Long-Term Potentiation.

13. The method of claim 1 wherein aiming comprises aiming a plurality of ultrasonic transducers to produce beams which intersect at a target.

14. The method of claim 1 where said at least one of ultrasound transducers delivers a defocused beam to alter the permeability of large volumes of a target in a brain.

15. The method of claim 1 wherein the ultrasound energy has a frequency in the range of 0.3 MHz to 0.8 MHz.

16. The method of claim 1 wherein the ultrasound energy is delivered at a power greater than 20 mW/cm² at a target tissue.

17. The method of claim 16 wherein the ultrasound energy is delivered at a power less than that causing tissue damage.

18. The method of claim 1 wherein the ultrasound energy has a stimulation frequency of lower than 500 Hz for inhibition of neural activity.

19. The method of claim 18 wherein the ultrasound energy has a pulse duration in the range from 0.1 to 20 msec repeated at frequencies of 2 Hz or lower for down regulation.

20. The method of claim 1 wherein the ultrasound energy has a stimulation frequency for excitation in the range of 500 Hz to 5 MHz.

21. The method of claim 20 wherein the ultrasound energy has a pulse duration in the range from 0.1 to 20 msec repeated at frequencies higher than 2 Hz for up regulation.

22. The method of claim 1 wherein the ultrasound has a focus area diameter in the range from 0.5 to 150 mm.

23. The method of claim 1 further comprising applying mechanical perturbations radially or axially to move the ultrasound transducers.
